(19) United States
(12) Patent Application Publication
(10) Pub. No.: US 2025/0041175 A1
(43) Pub. Date: Feb. 6, 2025

(54) COMPOSITION FOR SMOOTHING THE SKIN AND/OR THE LIPS

(71) Applicant: L V M H RECHERCHE, Saint Jean De Braye (FR)

(72) Inventors: Valérie ALARD, Saint Jean De Braye (FR); Ludivine LAURENT, Saint Jean De Braye (FR)

(73) Assignee: L V M H RECHERCHE, Saint Jean De Braye (FR)

(21) Appl. No.: 18/718,046

(22) PCT Filed: Dec. 8, 2022
(86) PCT No.: PCT/FR2022/052286

§ 371 Date: Jun. 9, 2024

(30) Foreign Application Priority Data
Dec. 10, 2021 (FR) FR2113303

(51) Int. Cl. A61K 8/34; A61Q 1/04; A61Q 19/08

(52) U.S. Cl. CPC A61K 8/345; A61Q 1/04; A61Q 19/08; A61K 2800/5922; A61K 2800/95

(57) ABSTRACT

Cosmetic process for smoothing the skin and/or the lips, for restoring volume to the face and/or the lips, and/or for reducing and/or filling wrinkles, comprising applying a composition comprising an aqueous phase and: a first cosmetic ingredient with one or more polyglycerols with 3-10 glycerol units and an average molecular weight of 200-600 g/mol, a second cosmetic ingredient which is different from the first with one or more polyglycerols with 3-10 glycerol units and an average molecular weight of 300-1000 g/mol, and optionally also a third cosmetic ingredient which is different from the first and from the second and comprises one or more polyglycerols which have 3-10 glycerol units and an average molecular weight of 200-1,000 g/mol, where the average molecular weight of the polyglycerols in the second cosmetic ingredient is at least 100 g/mol higher than the average molecular weight of the polyglycerols of the first cosmetic ingredient.

### FIELD OF THE INVENTION

The present invention concerns the cosmetic field and, in particular, compositions for smoothing the skin and/or the lips.

### STATE OF THE ART

Communication on anti-wrinkle effects with immediate and lasting skin smoothing (at least throughout the day) is a key asset and a strong driver of innovation and consumer satisfaction.

Currently, the immediate anti-wrinkle smoothing effect is supported, essentially, by the incorporation of contractile film-forming polymers, known for their tightening effect. However, the effectiveness of these polymers is limited, either by their durability (the polymer film cracks) and the lack of comfort, or by an ineffective smoothing effect (comfort/tightening effect paradox not achieved). Furthermore, to date, the smoothing effect provided by tightening polymers is insufficient compared to the current expectations of women and is not durable at a dose sensorially acceptable.

There therefore remains a need to develop alternatives to tightening polymers for an immediate and lasting smoothing result. The Applicant has demonstrated that the use of at least two cosmetic ingredients comprising one or more polyglycerols which have from 3 to 10 glycerol units, said cosmetic ingredients having average molecular weights which are different from each other by at least 100 g/mol, or even at least 200 g/mol, advantageously in combination with a polyol and possibly also a pro-penetrating agent, made it possible to meet this need. The polyglycerols and any other hydrophilic ingredients or active ingredients in the composition penetrate the first layers of the skin to provide volume and material to fill wrinkles “from the inside”, effectively and sustainably.

### DISCLOSURE OF THE INVENTION

A first subject-matter of the invention is therefore a cosmetic composition for care and/or makeup of the skin and/or the lips comprising, in a physiologically acceptable medium, an aqueous phase and:
a first cosmetic ingredient comprising one or more polyglycerols which have from 3 to 10 glycerol units, with an average molecular weight ranging from 200 to 600 g/mol,
a second cosmetic ingredient which is different from the first, and comprises one or more polyglycerols having from 3 to 10 glycerol units, with an average molecular weight ranging from 300 to 1000 g/mol,
and, optionally, also a third cosmetic ingredient, which is different from the first and the second, and comprises one or more polyglycerols which have from 3 to 10 glycerol units, with an average molecular weight ranging from 200 to 1000 g/mol,
with the proviso that the average molecular weight of the polyglycerols in the second cosmetic ingredient is at least 100 g/mol, or even at least 200 g/mol, higher than the average molecular weight of the polyglycerols of the first cosmetic ingredient.

The composition of the invention is a non-rinsed care composition, different from rinsed compositions which are hygiene or cleaning compositions.

Another subject-matter relates to a cosmetic process comprising the application to the skin and/or the lips of a composition as defined according to the invention, in particular to smooth the skin and/or the lips, and/or restore the volume of the face and/or the lips, provide firmness and/or elasticity, and/or reduce and/or fill in wrinkles.

The invention also concerns a non-therapeutic cosmetic use of at least:
a first cosmetic ingredient which comprises one or more polyglycerols which have from 3 to 10 glycerol units and have an average molecular weight ranging from 200 to 600 g/mol, and
a second cosmetic ingredient which is different from the first which comprises one or more polyglycerols which have from 3 to 10 glycerol units and have an average molecular weight ranging from 300 to 1000 g/mol,
as an active combination to smooth the skin, restore volume to the face and/or the lips, provide firmness and/or elasticity, and/or reduce and/or fill in wrinkles.

According to a particular and preferred embodiment, the invention relates to a cosmetic process for smoothing the skin and/or the lips, and/or restoring the volumes of the face and/or the lips, and/or reducing and/or filling in wrinkles comprising application to the skin and/or the lips of a composition as defined according to the invention.

The detailed description of the invention below describes the embodiments and compositions intended for the implementation of the cosmetic process according to the invention.

### DETAILED DESCRIPTION OF THE INVENTION

The present invention therefore concerns a cosmetic composition for care and/or makeup of the skin and/or the lips comprising, in a physiologically acceptable medium, an aqueous phase and:
a first cosmetic ingredient which comprises one or more polyglycerols which have from 3 to 10 glycerol units and have an average molecular weight ranging from 200 to 600 g/mol,
a second cosmetic ingredient which is different from the first and comprises one or more polyglycerols which have from 3 to 10 glycerol units and have an average molecular weight ranging from 300 to 1000 g/mol,
and, optionally, also a third cosmetic ingredient which is different from the first and from the second and comprises one or more polyglycerols which have from 3 to 10 glycerol units and have an average molecular weight ranging from 200 to 1,000 g/mol,
with the proviso that the average molecular weight of the polyglycerols in the second cosmetic ingredient is at least 100 g/mol, or even at least 200 g/mol, higher than the average molecular weight of the polyglycerols of the first cosmetic ingredient.

In particular it relates to a cosmetic process for smoothing the skin and/or the lips, and/or restoring the volumes of the face and/or the lips, and/or reducing and/or filling in wrinkles comprising the application onto the skin and/or the lips of a cosmetic care and/or makeup composition comprising, in a physiologically acceptable medium, an aqueous phase and:
a first cosmetic ingredient which comprises one or more polyglycerols which have from 3 to 10 glycerol units and have an average molecular weight ranging from 200 to 600 g/mol,
a second cosmetic ingredient which is different from the first and comprises one or more polyglycerols which have from 3 to 10 glycerol units and have an average molecular weight ranging from 300 to 1000 g/mol,
and optionally also a third cosmetic ingredient which is different from the first and from the second and comprises one or more polyglycerols which have from 3 to 10 glycerol units and have an average molecular weight ranging from 200 to 1,000 g/mol,
with the proviso that the average molecular weight of the polyglycerols in the second cosmetic ingredient is at least 100 g/mol, or even at least 200 g/mol, higher than the average molecular weight of the polyglycerols of the first cosmetic ingredient.

By “cosmetic composition” is meant any composition for cosmetic, that is to say, aesthetic purposes, which can be brought into contact with the superficial parts of the human body and more particularly with keratin materials, in particular human skin and/or lips. By skin is meant the skin of the face and/or neck and the skin of the body.

By “physiologically acceptable medium” is meant any excipient suitable for topical use, in contact with keratin materials, without risk of toxicity, incompatibility, instability and/or allergic response.

### Combination of Polyglycerols

The polyglycerols comprising from 3 to 10 glycerol units constituting the cosmetic ingredients based on polyglycerols which can be used according to the invention are in particular the following compounds:

$$POLYGLYCEROL - 3 ( 240.25 g / mol ) - CAS 56090 - 54 - 1 ,$$

$$POLYGLYCEROL - 4 ( 314.33 g / mol ) - CAS 56491 - 53 - 3$$

$$POLYGLYCEROL - 5 ( 380 g / mol ) ,$$

$$POLYGLYCEROL - 6 ( 462.5 g / mol ) - CAS 36675 - 34 - 0$$

$$POLYGLYCEROL - 10 ( 758.8 g / mol ) - CAS 9041 - 07 - 0.$$

The composition of the invention comprises at least two cosmetic ingredients each comprising one or more polyglycerols which have 3 to 10 glycerol units and, optionally, an aqueous solvent, in particular water.

According to a particular embodiment, each cosmetic ingredient based on polyglycerols consists essentially or even exclusively of one or more polyglycerols which have 3 to 10 glycerol units.

“Consisting essentially or exclusively of” means that the cosmetic ingredient based on polyglycerols comprises at least 95%, or better still 98%, or even better 99%, or even comprises 100% by weight of polyglycerols.

According to another particular embodiment, the cosmetic ingredient consists of one or more polyglycerols which have 3 to 10 glycerol units and a hydrophilic solvent, preferably water. The polyglycerol content in such a cosmetic ingredient comprising a hydrophilic solvent (e.g. water) will generally range from 70 to 90%, preferably from 80 to 90% by weight relative to the total weight of the cosmetic ingredient. The INCI name of such an ingredient would be: Polyglycerin-x and aqua (water) with x ranging from 3 to 10.

In the case where the cosmetic ingredients based on polyglycerols used according to the invention are mixtures of polyglycerols of different molecular weights, the term “average molecular weight” of said cosmetic ingredient means the average of the molecular weights of said polyglycerols taking into account their respective proportions in said ingredient, the value of this average molecular weight determining the corresponding INCI name, as explained below.

For example, the cosmetic ingredient marketed by the company Sakamoto under the name PGL-S includes polyglycerol-3 (majority), polyglycerol-4 and polyglycerol-5, and the average molecular weight of the molecular weights of the polyglycerols constituting this cosmetic ingredient is close to the molecular weight of polyglycerol-3. For this reason, the INCI name of this cosmetic ingredient, made up of a mixture of polyglycerols (polyglycerol-3, polyglycerol-4 and polyglycerol-5), is POLYGLYCERIN-3.

The expression “cosmetic ingredient with INCI name comprising POLYGLYCERIN-x with x ranging from 3 to 10”, therefore covers, according to the invention, a) a cosmetic ingredient with INCI name POLYGLYCERIN-x (without solvent) and b) a cosmetic ingredient with INCI name POLYGLYCERIN-x and aqua (water) (with solvent), in the case where the polyglycerol(s) are solubilized in a hydrophilic solvent, preferably water.

Thus, according to a particular embodiment, the composition of the invention comprises at least two cosmetic ingredients consisting of one or more polyglycerols which have from 3 to 10 glycerol units and optionally a hydrophilic solvent, with different INCI names, chosen from the group constituted by the INCI names comprising polyglycerin-3, polyglycerin-4, polyglycerin-6, polyglycerin-10, polyglycerin-6 and water, or polyglycerin-10 and water.

Advantageously, according to the invention, at least two cosmetic ingredients will be used consisting of one or more polyglycerols which have from 3 to 10 glycerol units, said ingredients having average molecular weights different from each other by at least 100 g/mol, or even at least 200 g/mol. The combined use of two cosmetic ingredients consisting essentially of one or more polyglycerols and having average molecular weights different from each other by at least 100 g/mol, or even at least 200 g/mol, makes it possible to have penetration kinetics prolonged over the time in the superficial layers of the skin, for an immediate effect and a lasting effect over time.

According to a preferred embodiment, the average molecular weight of the polyglycerols in the second cosmetic ingredient is at least 100 g/mol, or even at least 200 g/mol, higher than the average molecular weight of the polyglycerols of the first cosmetic ingredient.

Thus, the cosmetic composition for care and/or makeup of the skin and/or the lips comprises, in a physiologically acceptable medium, an aqueous phase and:
a first cosmetic ingredient which comprises one or more polyglycerols which have from 3 to 10 glycerol units and have an average molecular weight ranging from 200 to 600 g/mol, preferably from 200 to 500 g/mol,
a second cosmetic ingredient which is different from the first and comprises one or more polyglycerols which have from 3 to 10 glycerol units and have an average molecular weight ranging from 300 to 1000 g/mol, preferably from 400 to 800 g/mol.
and optionally also a third cosmetic ingredient which is different from the first and from the second and comprises one or more polyglycerols which have from 3 to 10 glycerol units and have an average molecular weight ranging from 200 to 1,000 g/mol,
with the proviso that the average molecular weight of the polyglycerols in the second cosmetic ingredient is at least 100 g/mol, or even at least 200 g/mol, higher than the average molecular weight of the polyglycerols of the first cosmetic ingredient.

According to a particular embodiment, the composition of the invention comprises an aqueous phase and:
a first cosmetic ingredient comprising one or more polyglycerols which have from 3 to 10 glycerol units, and whose INCI name comprises polyglycerin-3, or polyglycerin-4, or polyglycerin-6 or polyglycerin-10,
a second cosmetic ingredient comprising one or more polyglycerols which have 3 to 10 glycerol units, and whose INCI name comprises polyglycerin-4, or polyglycerin-6 or polyglycerin-10, different from the INCI name of the polyglycerols of the first cosmetic ingredient,
and, optionally, also a third cosmetic ingredient which is different from the first and from the second and comprises one or more polyglycerols which have from 3 to 10 glycerol units, and whose INCI name comprises polyglycerin-3, or polyglycerin-4, or polyglycerin-6 or polyglycerin-10.

For example, the composition of the invention comprises at least a first cosmetic ingredient with an INCI name comprising polyglycerin-3 and one or more other cosmetic ingredients with an INCI name comprising polyglycerin-4, or polyglycerin-6 or polyglycerin-6 and water, or polyglycerin-10 or polyglycerin-10 and water.

According to another example, the composition of the invention comprises at least one first cosmetic ingredient with the INCI name comprising polyglycerin-6 or polyglycerin-6 and water and one or more other cosmetic ingredients with the INCI name comprising polyglycerin-3, or polyglycerin-4, or polyglycerin-10 or polyglycerin-10 and water.

According to a particular embodiment, the composition of the invention comprises an aqueous phase and:
a first cosmetic ingredient with the INCI name comprising polyglycerin-3 and a second cosmetic ingredient with the INCI name comprising polyglycerin-6, or
a first cosmetic ingredient with the INCI name comprising polyglycerin-3 and a second cosmetic ingredient with the INCI name comprising polyglycerin-10, or
a first cosmetic ingredient with the INCI name comprising polyglycerin-6 and a second cosmetic ingredient with the INCI name comprising polyglycerin-10, or
a first cosmetic ingredient with the INCI name comprising polyglycerin-3, a second cosmetic ingredient with the INCI name comprising polyglycerin-6 and a third cosmetic ingredient with the INCI name comprising polyglycerin-10.

Thus, according to a particular and preferred embodiment, the composition of the invention will comprise a cosmetic ingredient with the INCI name polyglycerin-3 and a cosmetic ingredient with the INCI name polyglycerin-6 or polyglycerin-6 and water.

According to another particular and preferred embodiment, the composition of the invention will comprise a cosmetic ingredient with the INCI name polyglycerin-6 or polyglycerin-6 (and) water and a cosmetic ingredient with the INCI name polyglycerin-10 or polyglycerin-10 (and) water.

According to another particular and preferred embodiment, the composition of the invention will comprise a cosmetic ingredient with the INCI name polyglycerin-3 and a cosmetic ingredient with the INCI name polyglycerin-10 or polyglycerin-10 (and) water.

According to another particular and preferred embodiment, the composition of the invention will comprise a cosmetic ingredient with the INCI name polyglycerin-3, a cosmetic ingredient with the INCI name polyglycerin-6 or polyglycerin-6 (and) water and a cosmetic ingredient with the INCI name polyglycerin-10 or polyglycerin-10 (and) water.

Other combinations comprising at least two cosmetic ingredients with INCI names can be produced in the context of the invention as illustrated in the following Table 1 which describes two-by-two combinations (INCI name of each of the two cosmetic ingredients, symbolized by PG3 for Polyglycerin-3, PG4 for Polyglycerin-4, PG6 for Polyglycerin-6, PG10 for Polyglycerin-10):

Ingredient No. 2 (INCI name)

| | | PG3 | PG4 | PG6 | PG10 |
|---|---|---|---|---|---|
| Ingredient | PG3 | − | + | ++ | ++ |
| No. 1 (INCI | PG4 | + | − | + | ++ |
| name) | PG6 | ++ | + | − | ++ |
| | PG10 | ++ | ++ | ++ | − |

−: combination not preferred in the context of the invention because the difference between the average molecular weights of the polyglycerols between the two cosmetic ingredients is less than 100 g/mol.
+: combination according to the invention
++: preferred combination according to the invention

Other triple combinations falling within the scope of the invention are easily deducible from the double combinations described in Table 1 above, with the addition of a third cosmetic ingredient, which different from the first and second ingredients, and comprising one or more polyglycerols which have 3 to 10 glycerol units, and whose INCI name comprises polyglycerin-3 (PG3), polyglycerin-4 (PG4), polyglycerin-6 (PG6) or polyglycerin-10 (PG10).

As cosmetic ingredients based on polyglycerols usable according to the invention, the references marketed by the company SAKAMOTO can especially be named
PGL-S of INCI name POLYGLYCERIN-3
POLYGLYCERIN 310 of INCI name POLYGLYCERIN-4
POLYGLYCERIN-500 MB of INCI name POLYGLYCERIN-6 and AQUA (WATER)
POLYGLYCERIN-750 MB of INCI name POLYGLYCERIN-10 and AQUA (WATER)

The references marketed by the company SPIGA can also be named:
Pure Vegetal of INCI name Polyglycerin-3
Pure Vegetal of INCI name Polyglycerin-4
Pure Vegetal of INCI name Polyglycerin-6
Pure Vegetal of INCI name Polyglycerin-10

The total content of polyglycerols which have from 3 to 10 glycerol units in the composition of the invention will especially range from 0.5 to 20% by weight, preferably from 1 to 15% by weight relative to the total weight of the composition.

### Advantageous Additional Ingredients

The composition of the invention may also advantageously comprise additional agents different from the polyglycerols described above and chosen from the group consisting of moisturizing agents, pro-penetrating agents, smoothing or exfoliating agents, film-forming or tightening agents, antioxidant agents, gelling and/or structuring agents, fillers, pigments, and mixtures thereof.

According to a particular embodiment, the composition of the invention will comprise one or more polyol(s) different from the polyglycerols described above, as moisturizing agent, a pro-penetrating agent, and possibly also a smoothing agent or a film-forming agent, and mixtures thereof.

Polyols Different from the Polyglycerols Described Above

According to a particular embodiment of the invention, the composition further comprises at least one moisturizing agent, in particular at least one polyol different from the polyglycerols described above.

For the purposes of the present invention, “polyol” means any organic molecule comprising at least two free hydroxyl groups.

According to a particular form, the polyol can be chosen from sugars such as trehalose, mannitol, xylitol, sorbitol, and mixtures thereof.

According to a particular embodiment, the composition of the invention will comprise trehalose, sorbitol or a mixture thereof.

A polyol suitable for the invention may be a compound of the alkyl type, linear, branched or cyclic, saturated or unsaturated, bearing on the alkyl chain at least two-OH functions, in particular at least three-OH functions, and more particularly at least four-OH functions.

The polyols advantageously suitable for the formulation of a composition according to the present invention are C2-C8 glycols.

Advantageously, the C2-C8 glycol can, for example, be chosen from ethylene glycol, propylene glycol, 1,3 propanediol, butylene glycol, pentylene glycol, hexylene glycol, caprylyl glycol, glycerol (or glycerin), diglycerol (or diglycerin), and mixtures thereof.

According to a particular and preferred embodiment, the composition of the invention will comprise glycerol, diglycerol or a mixture thereof.

According to a preferred embodiment, the composition of the invention comprises at least glycerol, diglycerol or a mixture thereof and optionally also trehalose, sorbitol or a mixture thereof.

Thus, according to a preferred embodiment, the composition of the invention will also comprise at least one moisturizing agent, preferably at least one polyol different from the polyglycerols described above and chosen from the group consisting of sugars such as trehalose, mannitol, xylitol, sorbitol and mixtures thereof and C2-C8 glycols, such as ethylene glycol, propylene glycol, 1,3 propanediol, butylene glycol, pentylene glycol, hexylene glycol, caprylyl glycol, glycerol, diglycerol and mixtures thereof, preferably glycerol, diglycerol and a mixture thereof.

The total content of polyols, different from the polyglycerols described above, in the composition of the invention will generally range from 2 to 25% by weight, preferably from 5 to 20% by weight relative to the total weight of the composition.

The total content of glycerol and diglycerol in the composition of the invention will generally range from 2 to 12% by weight, preferably from 3 to 10% by weight relative to the total weight of the composition.

Agents promoting penetration into the skin (so-called ‘pro-penetrating’ agents)

According to another particular and advantageous embodiment, the composition of the invention will comprise a pro-penetrating agent.

According to the invention, “pro-penetrating” means a cosmetic agent making it possible to improve and/or promote the penetration of targeted cosmetic raw materials into the different layers of the skin.

Thus the composition of the invention will advantageously also comprise a pro-penetrating agent to improve and/or promote the penetration of the cosmetic ingredients and, in particular, of the polyglycerols and possible polyols of the composition, into the skin.

According to a particular embodiment, this pro-penetrating agent is chosen from the group consisting of:
Hydrophilic molecules chosen from urea and its derivatives, glycine or trimethylglycine (betaine); these small hydrophilic molecules penetrate the different layers of the skin, and thus promote the penetration of hydrophilic ingredients or active ingredients present in the composition (polyglycerols, polyols, etc.);
Fat solubilizing agents chosen, for example, from dimethyl isosorbide and ethanol; these agents solubilize the fatty substances in the intercellular cement and therefore promote the penetration of hydrophilic ingredients or active ingredients present in the composition (polyglycerols, polyols, etc.);
Surfactants, in particular non-ionic, preferably:
   glycerol surfactants such as esters of mono- or poly-glycerols and C6-C24 fatty acids, such as PG10-myristate and glyceryl stearate, and
   alkoxylated, preferably ethoxylated, C6-C24 fatty alcohols, such as the surfactants of INCI name Steareth-2 and Steareth-20;
      and mixtures thereof.

Preferably, the composition of the invention further comprises at least one pro-penetrating agent chosen from the group consisting of:
hydrophilic molecules chosen from urea and its derivatives, glycine, trimethylglycine (betaine),
   fat solubilizing agents chosen from dimethyl isosorbide and ethanol,
   glycerol-based surfactants such as esters of mono or polyglycerols and C6-C24 fatty acids, and alkoxylated, preferably ethoxylated, C6-C24 fatty alcohols, preferably glycerol-based surfactants such as esters of mono or polyglycerols and C6-C24 fatty acids;
   and mixtures thereof.

According to a preferred embodiment, the composition of the invention will comprise a pro-penetrating agent chosen from the group consisting of (i) hydrophilic molecules such as urea and betaine, (ii) non-ionic surfactants chosen from mono- or poly-glycerol esters and C6-C24 fatty acids and alkoxylated, preferably ethoxylated, C6-C24 fatty alcohols, and mixtures thereof.

More preferably, the composition of the invention will comprise a pro-penetrating agent chosen from the group consisting of (i) hydrophilic molecules such as urea and betaine, (ii) mono- or poly-glycerol esters and C6-C24 fatty acids, and mixtures thereof.

According to a particular and preferred embodiment, the composition of the invention will comprise a pro-penetrating agent chosen from urea, trimethylglycine (betaine), PG10-myristate and glyceryl stearate, or Steareth-2 and Steareth-20, and mixtures thereof.

More preferably, the composition of the invention will comprise a pro-penetrating agent chosen from urea, trimethylglycine (betaine), PG10-myristate and glyceryl stearate, and mixtures thereof.

The person skilled in the art will adapt the content of pro-penetrating ingredients according to their nature and according to the nature and content of the hydrophilic ingredients and active ingredients to be penetrated into the layers of the skin.

The total content of pro-penetrating agent in the composition of the invention will generally range from 1 to 20% by weight, preferably from 2 to 15% by weight, relative to the total weight of the composition.

The total content of hydrophilic molecules can generally range from 1 to 12% by weight, preferably from 2 to 10% by weight, relative to the total weight of the composition.

The total content of fat-solubilizing agents will generally range from 0.1 to 10% by weight, preferably from 0.5 to 5% by weight, relative to the total weight of the composition.

The total content of surfactant can generally range from 0.1 to 10% by weight, preferably from 0.5 to 5% by weight relative to the total weight of the composition.

### Smoothing or Exfoliating Agents

According to a particular embodiment, the composition for the skin also advantageously comprises a skin smoothing agent, in particular one or more hyaluronic acids or a salt thereof, and a mixture thereof; in particular the sodium, potassium or magnesium salts thereof, preferably sodium salt thereof.

Said hyaluronic acids can be in the form of a gel or in the form of a powder. The hyaluronic acid(s) (or salts thereof) used according to the invention may be crosslinked or not, and/or acetylated or not.

### Acetylated Hyaluronic Acids (or Salts Thereof)

According to a particular embodiment, the composition of the invention comprises at least one hyaluronic acid sodium salt chosen from a sodium salt of acetylated hyaluronic acid, a sodium salt of non-acetylated and non-cross-linked hyaluronic acid, a sodium salt of cross-linked hyaluronic acid, and mixtures thereof.

According to a particular and preferred embodiment, the composition of the invention comprises a sodium salt of acetylated hyaluronic acid.

Preferably, the acetylated hyaluronic acid used in the present invention is that described in patent application WO2018/162672.

In particular, said acetylated hyaluronic acid, or its sodium salt has an average degree of acetylation greater than 3.6, determined by quantitative 2D nuclear magnetic resonance (NMR).

According to a particular characteristic, the acetylated hyaluronic acid or its sodium salt used in the present invention has an average molecular weight of approximately 50 kDa or less, more particularly approximately 30 kDa or less, and a dispersion index less than 2.3, more particularly less than 2.0, more particularly less than 1.5.

In a preferred embodiment, the acetylated hyaluronic acid used in the present invention is a sodium salt of acetylated hyaluronic acid, in particular the product sold under the name “Primalhyal™ Ultrafiller” by the company Givaudan. The product “Primalhyal™ Ultrafiller” is referenced under the international nomenclature, INCI: SODIUM ACETYLATED HYALURONATE.

In particular, acetylated hyaluronic acid or its sodium salt, preferably the product marketed under the name “Primalhyal™ Ultrafiller”, is present in a content ranging from 0.01% to 10%, in particular from 0.05% to 5%, and especially from 0.1 to 1% by weight of raw material (product weight) relative to the total weight of the composition.

### Non-Acetylated and Non-Crosslinked Hyaluronic Acids (or Salts Thereof)

According to another particular embodiment, the composition comprises a sodium salt of hyaluronic acid which is neither acetylated nor crosslinked.

The product of INCI name SODIUM HYALURONATE marketed under the name HYALURONATE SODIUM HPM can especially be mentioned.

According to a particular embodiment, the non-crosslinked hyaluronic acid has a molecular weight less than or equal to 500 KDa, preferably less than or equal to 300 KDa or one of its salts.

According to a particular embodiment, the low molecular weight non-crosslinked hyaluronic acid used in the present invention is the product marketed under the name “Bashyal powder” by the company Soliance. The “Bashyal powder” product is a hyaluronic acid in powder form, 100% pure, i.e., composed of 100% active ingredient. The hyaluronic acid polymers used in the composition of this product have a molar mass ranging from 100 KDa to 300 KDa. “Bashyal powder” is referenced under the international nomenclature, INCI: SODIUM HYALURONATE.

According to another particular embodiment, the low molecular weight non-crosslinked hyaluronic acid used in the present invention is the product marketed under the name “Oligo HA” by the company Sochibo. The “Oligo HA” product is a hyaluronic acid in powder form, 100% pure, i.e., composed of 100% active ingredient. The hyaluronic acid polymers used in the composition of this product have a molar mass ranging from 5 KDa to 10 KDa. “Oligo HA” is referenced under the international nomenclature, INCI: HYALURONIC ACID.

According to the present invention, the molecular weight or molecular mass of hyaluronic acid means the “average” molecular mass (or average molecular weight) of the hyaluronic acid molecules constituting the raw material used. Actually, the raw materials used as sources of hyaluronic acids include hyaluronic acid polymers whose molar mass can vary slightly. These variations in molecular mass are notably related to the fact that the number of basic units (i.e., disaccharide monomers) included in the hyaluronic acid molecule at the time of its polymerization varies slightly. So, “molar mass of hyaluronic acid” or “average molar mass of hyaluronic acid”, must be understood to mean that the molar mass is “approximately” the indicated mass. In particular, the molar mass of hyaluronic acid indicated may vary by +20%, preferably +15% and even more preferably +10%.

### Crosslinked Hyaluronic Acids (or Salts Thereof)

According to a particular embodiment, the high molecular weight crosslinked hyaluronic acid used in the present invention is in the form of a gel. According to this particular embodiment, the high molecular weight crosslinked hyaluronic acid used in the present invention is preferably the product marketed under the name “Phylcare® Hyacross™ TL 100” by Bloomage Freda Biopharm Co., Ltd. “Phylcare® Hyacross™ TL 100” is a hyaluronic acid gel comprising 1% to 5% by weight of active ingredient relative to the total weight of the product. The hyaluronic acid polymers used in the composition of this product have a molar mass ranging from 1000 KDa to 1800 KDa. The product “Phylcare® Hyacross™ TL 100” is referenced under the International Nomenclature Cosmetic Ingredient (INCI): SODIUM HYALURONATE CROSSPOLYMER, PENTYLENE GLYCOL, AQUA.

According to another particular embodiment, the high molecular weight crosslinked hyaluronic acid used in the present invention is in the form of a powder. According to this particular embodiment, the high molecular weight crosslinked hyaluronic acid used in the present invention is preferably the product with the commercial name “Phylcare® Hyacross™ TL 200” produced by Bloomage Freda Biopharm Co., Ltd. “Phylcare® Hyacross™ TL 200” is a hyaluronic acid in powder form, 100% pure, that is to say composed of 100% active ingredient. The hyaluronic acid polymers used in the composition of this product have a molar mass greater than or equal to 3000 KDa. The product “Phylcare® Hyacross™ TL 200” is referenced under the international nomenclature, INCI: SODIUM HYALURONATE CROSSPOLYMER, Pentylene Glycol, Aqua.

According to a particular embodiment, the composition of the invention comprises a sodium salt of acetylated hyaluronic acid and a sodium salt of non-acetylated and non-crosslinked hyaluronic acid.

According to another particular embodiment, the composition comprises a crosslinked hyaluronic acid having a molecular weight greater than or equal to 1000 KDa, preferably greater than or equal to 2000 KDa or even greater than or equal to 3000 KDa or a salt thereof, and a non-crosslinked hyaluronic acid having a molecular weight less than or equal to 500 KDa, preferably less than or equal to 200 KDa or a salt thereof, and a mixture thereof.

The total content of hyaluronic acid(s) or a salt thereof may range from 0.01% to 10%, preferably from 0.05% to 5%, more preferably from 0.01% to 1% by weight of raw material (weight of product) relative to the total weight of the composition.

As another smoothing or exfoliating agent, the composition can also comprise alpha-hydroxy acids (AHAs).

### Film-Forming or Tightening Agents (Film-Forming Polymers)

According to a particular embodiment, the composition of the invention further comprises a film-forming agent or agent with a tightening effect on the surface of the skin, in particular a film-forming polymer.

“Agent with a tightening effect” means an agent capable of smoothing the skin on the surface to reduce wrinkles in the skin, through a mechanical effect linked to the elastic properties of the film formed on the surface of the skin.

The term “film-forming polymer” means a polymer capable of forming a continuous film on a substrate, from a solution, a dispersion or a suspension of this film-forming polymer, applied to said substrate.

The evaporation of the solvent in which it is dispersed allows this film-forming polymer to form a film on the surface of the skin and/or the lips and thus to prolong the effect provided by the composition on the restoration of the volumes of the face and/or the lips, and/or reduction and/or filling of wrinkles.

The film formed on the skin can also act as a reservoir for molecules dispersed in said film, which will gradually penetrate into the superficial layers of the skin and produce the desired cosmetic effect, for example a moisturizing effect.

These film-forming polymers can be natural or synthetic, fat-soluble or water-soluble.

Preferably, water-soluble polymers will be used, advantageously of natural origin.

Synthetic water-soluble polymers especially include vinyl polymers (e.g. polyvinyl alcohol), vinylpyrrolidone polymers and copolymers, polyacrylates, polyurethanes, latexes, and mixtures thereof.

Water-soluble polymers of natural origin especially include celluloses and derivatives thereof, polysaccharides especially chosen from pullulans, pectins, carrageenans, galactomannans, xanthans, alginates, starches, and mixtures thereof. The polysaccharides can also be in the form of an algae extract, in particular an extract of algae from the Laminariaceae family.

According to a particular and preferred embodiment, a pullulan will be used.

According to a particular embodiment, a polysaccharide of natural origin having a molecular weight of less than 600,000 Daltons, preferably less than 400,000 Daltons and even more preferably less than 200,000 Daltons can be mentioned, in particular in the form of an extract of Laminaria flexicaulis as described in the Applicant's application FR 2 828 810.

According to another particular method, a starch, optionally modified, can be used. The starch is preferably a native starch, especially chosen from corn starches, pea starches, tapioca starches, potato starches, rice starches, cassava starches and wheat starches. Preferably tapioca starch will be used.

Starches are especially described in the Applicant's application FR 2 990 348.

According to a particular embodiment, the composition comprises a film-forming polymer chosen from the group consisting of synthetic polymers or polymers of natural origin, in particular vinyl polymers, polyacrylates, polyurethanes, celluloses and derivatives thereof, polysaccharides and derivatives thereof, and mixtures thereof.

The total content of film-forming polymer(s) when they are present in the composition of the invention will generally range from 0.1 to 10% by weight, preferably from 0.2 to 5% by weight relative to the total weight of the composition.

Thus, according to a particular embodiment of the invention, the composition of the invention may comprise, in addition to the two cosmetic ingredients based on polyglycerols:
a polyol, preferably a C2-C8 glycol, preferably a glycerol and optionally also a diglycerol;
a pro-penetrating agent, preferably chosen from urea, trimethylglycine (betaine), non-ionic surfactants of the ester type of mono- or poly-glycerols and C6-C24 fatty acids, such as PG10-myristate and glyceryl stearate, or alkoxylated, preferably ethoxylated, C6-C24 fatty alcohols, and mixtures thereof;
a film-forming polymer, preferably chosen from a vinyl polymer, a polyacrylate, a polyurethane, a polysaccharide and mixtures thereof;
a smoothing agent, preferably chosen from hyaluronic acid or a salt thereof, or AHAs; and mixtures thereof.

According to a particular embodiment, the cosmetic composition for care and/or makeup of the skin and/or lips according to the invention comprises, in a physiologically acceptable medium, an aqueous phase and:
a first cosmetic ingredient which comprises one or more polyglycerols which have from 3 to 10 glycerol units and have an average molecular weight ranging from 200 to 600 g/mol,
a second cosmetic ingredient which is different from the first and comprises one or more polyglycerols which have from 3 to 10 glycerol units and have an average molecular weight ranging from 300 to 1000 g/mol,
and, optionally, also a third cosmetic ingredient which is different from the first and from the second and comprises one or more polyglycerols which have from 3 to 10 glycerol units and have an average molecular weight ranging from 200 to 1,000 g/mol,
   with the proviso that the average molecular weight of the polyglycerols in the second cosmetic ingredient is at least 100 g/mol, or even at least 200 g/mol, higher than the average molecular weight of the polyglycerols of the first cosmetic ingredient.
and, in addition, a pro-penetrating agent chosen from the group consisting of:
   hydrophilic molecules chosen from urea and its derivatives, glycine, trimethylglycine (betaine),
   fat solubilizing agents chosen from dimethyl isosorbide and ethanol,
   glycerol-based surfactants such as esters of mono or polyglycerols and C6-C24 fatty acids, and alkoxylated, preferably ethoxylated, C6-C24 fatty alcohols, preferably glycerol-based surfactants such as esters of mono or polyglycerols and C6-C24 fatty acids;
   and mixtures thereof.

According to a particular and preferred embodiment, the pro-penetrating agent is chosen from hydrophilic molecules chosen from urea and its derivatives, glycine and trimethylglycine (betaine).

According to the particular preferred embodiments, the cosmetic composition according to the invention comprises:
a first cosmetic ingredient with the INCI name comprising polyglycerin-3 and a second cosmetic ingredient with the INCI name comprising polyglycerin-6, or
a first cosmetic ingredient with the INCI name comprising polyglycerin-3 and a second cosmetic ingredient with the INCI name comprising polyglycerin-10, or
a first cosmetic ingredient with the INCI name comprising polyglycerin-6 and a second cosmetic ingredient with the INCI name comprising polyglycerin-10, or
a first cosmetic ingredient with the INCI name comprising polyglycerin-3, a second cosmetic ingredient with the INCI name comprising polyglycerin-6 and a third cosmetic ingredient with the INCI name comprising polyglycerin-10.

Thus, according to a particular and preferred embodiment, the composition of the invention comprises:
a first cosmetic ingredient with the INCI name comprising polyglycerin-3 and a second cosmetic ingredient with the INCI name comprising polyglycerin-6, and
a pro-penetrating agent chosen from hydrophilic molecules chosen from urea and its derivatives, glycine and trimethylglycine (betaine), preferably betaine.

The composition of this particular and preferred embodiment may further comprise, as film-forming agent, a water-soluble polymer of natural origin, and in particular a pullulan.

According to another particular and preferred embodiment, the composition of the invention comprises:
a first cosmetic ingredient with the INCI name comprising polyglycerin-6 and a second cosmetic ingredient with the INCI name comprising polyglycerin-10, and
a pro-penetrating agent chosen from hydrophilic molecules chosen from urea and its derivatives, glycine and trimethylglycine (betaine), preferably urea.

### Fatty Compounds

The composition of the invention may also comprise an oily phase comprising oils and possibly fatty compounds, such as waxes, pasty compounds and mixtures thereof. These include waxes of plant origin, butters and triglycerides of fatty acids and derivatives thereof.

### Fillers

The composition may also include a filler.

“Fillers” mean particles of plate, spherical or oblong shape, colourless or white, mineral or organic, natural or synthetic, which are in an insoluble form and dispersed in the medium of the composition. These fillers especially serve to modify the rheology or texture of the composition and/or to confer a mattifying effect. “Blurring effect” filler means an effect which camouflages the micro-reliefs of the skin and thus makes it possible to reduce, through an optical correction effect, imperfections in relief and/or colour, such as wrinkles, fine lines, pores. and spots.

Silica, cellulose, boron nitride, lauroyl lysine and mixtures thereof can especially be mentioned.

### Formulations

The cosmetic composition of the invention is a composition for the care and/or makeup of keratin materials, in particular of the skin and/or the lips, preferably the skin of the face and/or neck, and/or the lips.

The composition of the invention is a non-rinsed composition, different from rinsed compositions which are hygiene or cleaning compositions.

According to a particular embodiment, the care and/or makeup composition for the skin and/or the lips is in the form of a lotion, a gel or a cream, for example a water-in-oil emulsion or oil-in-water emulsion, or a serum, preferably in the form of a gelled aqueous phase.

The composition may especially have a liquid, fluid or thick consistency.

According to a particular embodiment, the composition is a care composition for the skin and/or the lips.

According to a particular embodiment, the composition is a makeup composition for the skin and/or the lips.

This will especially involve skin care, in particular anti-aging facial skin care or a makeup composition for the skin such as a smoothing foundation for facial skin. According to another embodiment, it could be a lip treatment, in particular a smoothing and/or volumizing lip treatment or a lip makeup composition such as a lipstick with smoothing and/or volumizing effect.

### Cosmetic Process

The invention also relates to a cosmetic process comprising the application to the skin and/or the lips of a composition according to the invention, in particular to smooth the skin, restore the volumes of the face and/or the lips, and/or reduce and/or fill in wrinkles.

Thus, according to a particular and preferred embodiment, the invention relates to a cosmetic process for smoothing the skin and/or the lips, and/or restoring the volumes of the face and/or the lips, and/or reducing and/or filling in wrinkles comprising the application to the skin and/or the lips of a composition as defined according to the invention.

The detailed description of the invention below describes the embodiments and compositions intended for the implementation of the cosmetic process according to the invention.

The composition of the invention is applied to healthy skin and/or lips, i.e., not presenting conditions or disorders which would result from a pathological state (“unhealthy” subjects, with a pathology). The terms healthy skin and/or lips or skin and/or lips will be used interchangeably in the rest of the description.

The composition also promotes hydration of the skin and/or lips.

According to a particular embodiment, the compositions of the invention will be applied to healthy subjects presenting a loss of volume in the face.

According to another particular embodiment, the compositions of the invention will be applied to healthy subjects presenting signs related to skin aging, in particular wrinkles.

According to another particular embodiment, the compositions of the invention will be applied to healthy subjects with dry skin.

According to a particular embodiment, the compositions of the invention will be applied to healthy subjects presenting a loss of firmness and/or elasticity of the skin.

According to a particular embodiment, the compositions of the invention will be applied to healthy subjects having dry lips and/or thin lips and/or lips presenting lines.

### Cosmetic Use

The present invention also concerns a non-therapeutic cosmetic use of at least:
a first cosmetic ingredient which comprises one or more polyglycerols which have from 3 to 10 glycerol units and have an average molecular weight ranging from 200 to 600 g/mol, and
a second cosmetic ingredient which is different from the first and comprises one or more polyglycerols which have from 3 to 10 glycerol units and have an average molecular weight ranging from 300 to 1000 g/mol,
   as an active combination to smooth the skin, restore volume to the face and/or the lips, provide firmness and/or elasticity, and/or reduce and/or fill in wrinkles.

According to a particular embodiment, the active combination according to the invention further comprises a pro-penetrating agent chosen from the group consisting of:
hydrophilic molecules chosen from urea and its derivatives, glycine and trimethylglycine (betaine),
fat solubilizing agents chosen from dimethyl isosorbide and ethanol,
glycerol-based surfactants such as esters of mono or polyglycerols and C6-C24 fatty acids, and alkoxylated, preferably ethoxylated, C6-C24 fatty alcohols, preferably glycerol-based surfactants such as esters of mono or polyglycerols and C6-C24 fatty acids;
and mixtures thereof.

The active combination will also advantageously comprise a film-forming agent, in particular a water-soluble polymer of natural origin, and in particular a pullulan.

The invention will be illustrated in the following non-limiting examples. Unless otherwise indicated, the % are expressed by weight relative to the total weight of the composition.

### EXAMPLES

In the examples that follow, the following INCI name ingredients have been used:
POLYGLYCERIN-3 marketed under the name PGL-S by SAKAMOTO
POLYGLYCERIN-4 marketed under the name POLYGLYCERIN 310 by SAKAMOTO
POLYGLYCERIN-6 marketed under the name POLYGLYCERIN-500 MB by SAKAMOTO
POLYGLYCERIN-10 marketed under the name POLYGLYCERIN-750 MB by SAKAMOTO.

### Example 1: Kinetics of Penetration of Polyglycerols into the Superficial Layers of the Skin

A preliminary study (unpublished) was carried out on sections of human skin studied by imaging (7T-MALDI-FT-ICR mass spectrometry imaging (MSI)) and made it possible to demonstrate the interest of using polyglycerols or mixtures of polyglycerols on the skin. By an image-taking method, this study especially showed that the penetration speed of polyglycerols and the depth of penetration at the surface layers of the skin (epidermis and superficial dermis) are influenced by the molecular weight of these compounds, i.e., the size of the glycerol chain.

In practice, it has been demonstrated that polyglycerol-4 penetrates more quickly but is stationed in the superficial dermis, compared to polyglycerol-3 which penetrates rapidly and reaches the superficial dermis very quickly. Polyglycerol-5 and -6 penetrate less deeply than polyglycerol-3 or polyglycerol-4.

It follows from these data that it is particularly interesting to combine polyglycerols of different molecular weights in cosmetic compositions, in particular of average molecular weights different from each other by at least 100 g/mol, or even at least 200 g/mol, to obtain an effect both on the kinetics of skin penetration and on the depth of penetration into the superficial layers of the skin.

These compounds can act at different levels of the superficial layers of the skin and more or less quickly, and produce their effect, especially by moisturizing and/or smoothing by swelling or filling the superficial layers of the skin, with a lasting effect by means of controlled penetration kinetics.

Combinations of polyglycerols are particularly preferred in cosmetic compositions comprising an aqueous phase, such as gelled aqueous compositions or emulsion type compositions, in which the aqueous phase is dispersed in a fatty phase (water-in-oil emulsion) or constitutes the continuous phase (oil-in-water emulsion).

### Example 2—Effect of the Combination of the Invention on Skin Smoothing

In this example, the effect of the combination of the invention compared to a control formula was studied.

The composition according to the invention comprises glycerol, a mixture of polyglycerols and a pro-penetrating agent (urea).

The formulas of the two compositions tested are described in Table 2 below (percentages expressed by weight of ingredient relative to the final formula):

| | Ingredients (INCI names) | Control | Invention |
|---|---|---|---|
| A | Polyglycerin-6 (POLYGLYCERIN 500 MB) | — | 6 |
| A | Polyglycerin-10 (POLYGLYCERIN 750 MB) | — | 6 |
| A | Urea (pro-penetrating agent) | — | 5 |

| | | |
|---|---|---|
| A | Glycerol | 5 |
| A | Diglycerol | 1 |
| A | Acetylated sodium hyaluronate | 0.30 |
| | (Primalhyal ™ Ultrafiller) | |
| A | Sodium hyaluronate | 0.05 |
| A | Butylene glycol and Pentylene glycol | 8.5 |
| B | Hydrogenated lecithin | 0.5 |
| C | Vegetable oils | 7 |
| D | Hydroxyethyl Acrylate/Sodium | 0.8 |
| | Acryloyldimethyl Taurate Copolymer | |
| E | Boron nitride | 0.5 |
| F | Preservatives | Qs |
| | Water | Qs 100 |

The composition is prepared according to the process below:

Prepare the quantity of water in a beaker and heat it to 40° C.

Incorporate phase D and stir until it is homogenized, and a gel forms.

Add phase A with stirring, then phase B.

Prepare phase C in another beaker, and heat to 40° C.

Add phase C to the aqueous phase with vigorous stirring to form the emulsion.

Stop heating then add E then F while stirring, until a homogeneous mixture is obtained.

Smoothing effect after 4 hours.

### 1—Taking an Impression

The study method is based on making replicas (or impressions) of a skin area of interest.

In this case, the area studied is that of the corner of the eye, also called “crow's feet”.

A first impression is taken before application of the composition studied (CONTROL or INVENTION).

To make the impression, an adhesive ring measuring 24 mm in diameter is applied to the test area. Using a spatula, a Silflo silicone casting resin (Flexico) plus a catalyst (Flexico) were then spread inside the ring. After drying, the replica is peeled off from the surface of the skin.

A second impression is made four hours after application of the cosmetic composition (control or invention). The protocol is reproduced for each cosmetic composition in the study.

### 2—Image Analysis

Each replica is positioned on a stand and illuminated using a grazing illuminator with an angle of incidence of 35 degrees (35) in order to generate shadows behind each line of the skin surface.

These shadows are then analysed on the video images of the replica. A MONADERM video imaging system was used, allowing the acquisition of high resolution images of the impression made and the digital values representing the shades of grey of each point (pixel) analysed on the images.

The replicas taken after application of the cosmetic composition tested are strictly repositioned in relation to those taken before application, corresponding to T0.

The QUANTITRIDES software calculates the following parameters: number of wrinkles, total surface area of wrinkles in mm²; total length of wrinkles in mm; mean length of wrinkles in mm; mean depth of wrinkles in μm.

The smoothing effect itself is evaluated by comparing these parameters before and after application. If the smoothing effect is significant, a reduction is observed in the wrinkle zones, their length and/or their surface area or depth.

For this example, the test was carried out on a number of volunteers equal to 16. (n=16).

A quantity of the formula corresponding to 2 microlitres per square centimetre (μl/cm²) was applied to the area tested. An image was taken at TO (before application) then 4 hours after application of the product to the area studied.

The results, after 4 hours, are presented in Table 3 below:

| Parameters evaluated | Control | Invention |
|---|---|---|
| Reduction in the number of wrinkles | − | +++ |
| Reduction in the total surface area of wrinkles | − | +++ |
| Reduction in the total length of wrinkles | − | +++ |

+++: significant reduction (−10% to −20%), presence of a smoothing effect
−: no significant reduction, absence of smoothing effect

For the composition of the invention, four hours after application, a significant reduction in the number of wrinkles, the total surface area of the wrinkles and the total length of the wrinkles is observed.

For the control, no smoothing effect was measured four hours after application of the formula.

This demonstrates the lasting smoothing effect produced by the composition of the invention compared to the control formula tested.

### Example 3: Smoothing Effect Optimized by the Addition of a Film-Forming Agent to the Combination of the Invention

In this example, the effect of a cream according to the invention was studied in relation to a control formula.

The composition according to the invention comprises glycerol, a mixture of polyglycerols, a pro-penetrating agent (trimethylglycine, also called betaine) and a film-forming agent (pullulan).

The contents of polyglycerols and pro-penetrating agent are lower than those used in Example 2 above, and the formula of the invention also contains a pullulan film-forming agent.

The formulas of the two compositions tested are described in Table 4 below (percentages expressed by percentage relative to the final formula):

| Ingredients (INCI names) | Control | Invention |
|---|---|---|
| Polyglycerin-3 (PGL-S MB) | — | 3 |
| Polyglycerin-6 (POLYGLYCERIN 500 MB) | — | 2 |
| Pullulan (film-forming agent) | — | 2 |

| | |
|---|---|
| Trimethylglycine (pro-penetrating agent) | 1 |
| Glycerol | 5.05 |
| Glycols | 8 |
| Acetylated sodium hyaluronate | 0.30 |
| (Primalhyal ™ Ultrafiller) | |
| Sodium hyaluronate | 0.05 |
| Hydroxyethyl Acrylate/Sodium | 0.8 |
| Acryloyldimethyl Taurate Copolymer | |
| Hydrogenated lecithin | 0.5 |
| Vegetable oils | 7.0 |
| Preservatives | Qs |
| Boron nitride | 0.5 |
| Water | Qs 100 |

Smoothing Effect after 4 Hours (n=13)

The test is carried out according to the method described in Example 2, on a number of volunteers n equal to 13.

The results, after 4 hours, are presented in Table 5 below:

| | Control | Invention |
|---|---|---|
| Parameters evaluated | formula | formula |
| Reduction in the number of wrinkles | − | ++++ |
| Reduction in the total surface area of wrinkles | − | ++++ |
| Reduction in the total length of wrinkles | − | ++++ |

++++: significantly improved reduction (−22% to −30%), presence of an optimized smoothing effect due to the presence of the film-forming agent
−: no significant reduction, absence of smoothing effect

The composition of the invention produces a significant improved smoothing effect, due the presence of the film-forming agent (pullulan) combined with the combination of polyglycerols, on the wrinkles around the eye four hours after application, compared to T0.

The control formula shows no significant smoothing effect on wrinkles in the same study area four hours after application, compared to T0.

### Example 4: Fluid Cream According to the Invention

The composition below is a fluid cream in the form of a gelled aqueous oil-in-water emulsion, comprising a combination of polyglycerols of different molecular weights, glycerol and pro-penetrating agents (urea, PG-10 myristate and glyceryl stearate). The percentages are expressed as a weight percentage relative to the final formula (Table 6).

| Ingredients (INCI names) | % |
|---|---|
| Polyglycerin-3 (PGL-S) | 6 |
| Polyglycerin-6 (POLYGLYCERIN 500 MB) | 5.4 |
| Polyglycerin-10 (POLYGLYCERIN 750 MB | 5.4 |
| Glycerol | 6 |
| Urea (pro-penetrating agent) | 10 |
| Vegetable oil | 10 |
| Plant triglyceride | 5 |
| Fatty acids | 3 |
| Glycerol ester (PG-10 myristate) | 5 |
| (pro-penetrating agent) | |
| Glyceryl stearate (pro-penetrating agent) | 0.5 |
| Sodium Stearoyl Glutamate | 0.2 |
| Xanthan gum | 0.1 |
| Acetylated sodium hyaluronate | 0.30 |
| (Primalhyal ™ Ultrafiller) | |
| Sodium hyaluronate | 0.05 |
| Pentylene Glycol | 0.5 |
| Preservatives | qs |
| pH adjuster | qs |
| Water | Qs 100 |

The composition of Table 6 above is tested according to the method described in Example 2 on a number of volunteers equal to 15.

The composition has a smoothing action on wrinkles in the corner of the eye area. The number of wrinkles and the total length of wrinkles decreased significantly (from −15% to −20%), four hours after application of the composition to the area studied.

### Example 5: Thick Cream According to the Invention

The composition below is a thick eye contour cream in the form of an oil-in-water emulsion, comprising a combination of polyglycerols of different molecular weights, glycerol and pro-penetrating agents (urea, steareth-21 and steareth-2).

The formula is detailed in Table 7 below. The percentages are expressed by weight of the final composition.

| Ingredients (INCI names) | % |
|---|---|
| Polyglycerin-3 (PGL-S) | 6 |
| Polyglycerin-6 (POLYGLYCERIN 500 MB) | 5.4 |
| Polyglycerin-10 (POLYGLYCERIN 750 MB) | 5.4 |
| Glycerol | 3.9 |
| Urea (pro-penetrating agent) | 10 |
| Polyurethane-35 (Film-forming agent) | 4 |
| Oils | 18.0 |
| Stearyl Alcohol | 1.3 |
| Steareth-21 (pro-penetrating agent) | 1 |
| Steareth-2 (pro-penetrating agent) | 0.5 |
| Waxes | 0.5 |
| Preservatives | qs |
| Acrylates/C10-C30 Alkyl Acrylate Crosspolymer | 0.3 |
| Xanthan gum | 0.1 |
| Acetylated sodium hyaluronate | 0.1 |
| (Primalhyal ™ Ultrafiller) | |
| Sodium hyaluronate | 0.1 |
| Sodium Hydroxide | 0.05 |
| Glycols | 3.6 |
| Water | Qs 100 |

The composition of Table 7 above is tested according to the method described in Example 2 on a number of volunteers equal to 15.

The composition has a smoothing action on wrinkles in the corner of the eye area. The total surface area of wrinkles and the total length of wrinkles decreased significantly (from −15% to −20%), four hours after application of the composition to the area studied.

A comparative study of the formulas of Examples 4 and 5 on sections of human skin studied by imaging (7T-MALDI-FT-ICR mass spectrometry imaging (MSI)) made it possible to show that the polyglycerols of formula 4 penetrate into the layers of the skin better than the polyglycerols of formula 5, in favour of the use, as a pro-penetrating agent, of (poly)glycerol-based surfactants (glyceryl stearate and PG-10 myristate) rather than ethoxylated fatty alcohol surfactants (steareth-2 and steareth-21).

### Example 6: Composition for the Lips

The composition below is a composition for the lips, comprising a combination of polyglycerols of different molecular weights, glycerol and smoothing agents (hyaluronic acids).

The formula is detailed in Table 8 below. The percentages are expressed by weight of the final composition.

| | Content |
|---|---|
| Ingredients | (%) |
| Polyglycerin-3 (PGL-S) | 1.0 |
| Polyglycerin-6 (POLYGLYCERIN 500 MB) | 4.5 |
| Vegetable oil | 38 |
| Hydroxystearic acid | 1.2 |
| Gelling agent (bentone) | 13 |
| Vegetable wax | 5 |
| Cellulose | 2 |
| Glycerol | 10 |
| Carbopol | 0.2 |
| Sodium hyaluronate | 0.5 |
| Acetylated sodium hyaluronate | 0.3 |
| (Primalhyal ™ Ultrafiller) | |
| Preservatives | 0.3 |
| Water | QS 100 |

Applied to the lips and the area around the lips, this composition gives a smoothing and plumping effect to the lips and the area surrounding them.

### Example 7: Skin Smoothing Composition

The composition below is a fluid cream in the form of a gelled aqueous oil-in-water emulsion, comprising a combination of polyglycerols of different molecular weights, glycerol and pro-penetrating agents (urea, PG-10 myristate and glyceryl stearate). The percentages are expressed as a weight percentage relative to the final formula (Table 9).

| Ingredients (INCI names) | % |
|---|---|
| Polyglycerin-3 (PGL-S) | 2.25 |
| Polyglycerin-6 (POLYGLYCERIN 500 MB) | 0.68 |
| Glycerol | 5 |
| Betaine (=trimethylglycine, | 3 |
| pro-penetrant agent) | |
| Vegetable oil | 10 |
| Plant triglyceride | 5 |
| Fatty acids | 3 |
| Glycerol ester (PG-10 myristate) | 2 |
| (pro-penetrating agent) | |
| Glyceryl stearate (pro-penetrating agent) | 0.5 |
| Sodium Stearoyl Glutamate | 0.2 |
| Xanthan gum | 0.1 |
| Pentylene Glycol | 0.5 |
| Preservatives | Qs |
| pH adjuster | Qs |
| Water | Qs 100 |

The composition has a smoothing action on wrinkles in the corner of the eye area.

The penetration of polyglycerols (PG3, PG6) is facilitated by the combination of pro-penetrating agents (glycerol esters and betaine).

1. A cosmetic process for smoothing the skin and/or the lips, and/or restoring the volumes of the face and/or the lips, and/or reducing and/or filling in wrinkles comprising the application onto the skin and/or the lips of a cosmetic care and/or makeup composition comprising, in a physiologically acceptable medium, an aqueous phase and:
a first cosmetic ingredient which comprises one or more polyglycerols which have from 3 to 10 glycerol units and have an average molecular weight ranging from 200 to 600 g/mol, and
a second cosmetic ingredient which is different from the first and comprises one or more polyglycerols which have from 3 to 10 glycerol units and have an average molecular weight ranging from 300 to 1000 g/mol,
with the proviso that the average molecular weight of the polyglycerols in the second cosmetic ingredient is at least 100 g/mol, or even at least 200 g/mol, higher than the average molecular weight of the polyglycerols of the first cosmetic ingredient.

2. The cosmetic process according to claim 1, wherein the composition comprises:
a first cosmetic ingredient comprising one or more polyglycerols which have from 3 to 10 glycerol units, and whose INCI name comprises polyglycerin-3, or polyglycerin-4, or polyglycerin-6 or polyglycerin-10, and
a second cosmetic ingredient comprising one or more polyglycerols which have 3 to 10 glycerol units, and whose INCI name comprises polyglycerin-4, or polyglycerin-6 or polyglycerin-10, different from the INCI name of the polyglycerols of the first cosmetic ingredient.

3. The cosmetic process according to claim 1,
wherein the composition comprises:
a first cosmetic ingredient with the INCI name comprising polyglycerin-3 and a second cosmetic ingredient with the INCI name comprising polyglycerin-6, or
a first cosmetic ingredient with the INCI name comprising polyglycerin-3 and a second cosmetic ingredient with the INCI name comprising polyglycerin-10, or
a first cosmetic ingredient with the INCI name comprising polyglycerin-6 and a second cosmetic ingredient with the INCI name comprising polyglycerin-10.

4. The cosmetic process according to claim 1, wherein the composition is characterized in that the total content of polyglycerols which have from 3 to 10 glycerol units ranges from 0.5 to 20% by weight relative to the total weight of the composition.

5. The cosmetic process according to claim 1, wherein the composition also comprises at least one moisturizing agent.

6. The cosmetic process according to claim 1, wherein the composition also comprises a pro-penetrating agent to improve and/or promote the penetration of cosmetic ingredients into the skin.

7. The cosmetic process according to claim 1, wherein the composition also comprises a skin smoothing agent.

8. The cosmetic process according to claim 1, wherein the composition also comprises a film-forming polymer.

9. The cosmetic process according to claim 1, wherein the care and/or makeup composition for the skin and/or the lips is in the form of a lotion, a gel, a cream, or a serum.

10.-11. (canceled)

12. A cosmetic composition for care and/or makeup of the skin and/or the lips comprising, in a physiologically acceptable medium, an aqueous phase and:
a first cosmetic ingredient which comprises one or more polyglycerols which have from 3 to 10 glycerol units and have an average molecular weight ranging from 200 to 600 g/mol, and
a second cosmetic ingredient which is different from the first and comprises one or more polyglycerols which have from 3 to 10 glycerol units and have an average molecular weight ranging from 300 to 1000 g/mol,
with the proviso that the average molecular weight of the polyglycerols in the second cosmetic ingredient is at least 100 g/mol, or even at least 200 g/mol, higher than the average molecular weight of the polyglycerols of the first cosmetic ingredient
and, in addition, a pro-penetrating agent chosen from the group consisting of:
hydrophilic molecules chosen from urea and its derivatives, glycine, trimethylglycine (betaine),
fat solubilizing agents chosen from dimethyl isosorbide and ethanol,
glycerol-based surfactants;
and mixtures thereof.

13. The composition according to claim 12, wherein the pro-penetrating agent is chosen from hydrophilic molecules chosen from urea and its derivatives, glycine and trimethylglycine (betaine).

14. The cosmetic composition according to claim 12, wherein in that it comprises:
a first cosmetic ingredient with the INCI name comprising polyglycerin-3 and a second cosmetic ingredient with the INCI name comprising polyglycerin-6, or
a first cosmetic ingredient with the INCI name comprising polyglycerin-3 and a second cosmetic ingredient with the INCI name comprising polyglycerin-10, or
a first cosmetic ingredient with the INCI name comprising polyglycerin-6 and a second cosmetic ingredient with the INCI name comprising polyglycerin-10, or
a first cosmetic ingredient with the INCI name comprising polyglycerin-3, a second cosmetic ingredient with the INCI name comprising polyglycerin-6 and a third cosmetic ingredient with the INCI name comprising polyglycerin-10.

15. The cosmetic composition according to claim 14, wherein it comprises:
a first cosmetic ingredient with the INCI name comprising polyglycerin-3 and a second cosmetic ingredient with the INCI name comprising polyglycerin-6, and a pro-penetrating agent chosen from hydrophilic molecules chosen from urea and its derivatives, glycine and trimethylglycine (betaine), or
a first cosmetic ingredient with the INCI name comprising polyglycerin-6 and a second cosmetic ingredient with the INCI name comprising polyglycerin-10, and a pro-penetrating agent chosen from hydrophilic molecules chosen from urea and its derivatives, glycine and trimethylglycine (betaine).

16. The cosmetic process according to claim 1, wherein the cosmetic care and/or makeup composition also comprises a third cosmetic ingredient which is different from the first and from the second and comprises one or more polyglycerols which have from 3 to 10 glycerol units and have an average molecular weight ranging from 200 to 1,000 g/mol.

17. The cosmetic process according to claim 16, wherein the composition comprises:
a first cosmetic ingredient comprising one or more polyglycerols which have from 3 to 10 glycerol units, and whose INCI name comprises polyglycerin-3, or polyglycerin-4, or polyglycerin-6 or polyglycerin-10,
a second cosmetic ingredient comprising one or more polyglycerols which have 3 to 10 glycerol units, and whose INCI name comprises polyglycerin-4, or polyglycerin-6 or polyglycerin-10, different from the INCI name of the polyglycerols of the first cosmetic ingredient, and
a third cosmetic ingredient which is different from the first and from the second and comprises one or more polyglycerols which have from 3 to 10 glycerol units, and whose INCI name comprises polyglycerin-3, or polyglycerin-4, or polyglycerin-6 or polyglycerin-10.

18. The cosmetic process according to claim 16, wherein the composition comprises a first cosmetic ingredient with the INCI name comprising polyglycerin-3, a second cosmetic ingredient with the INCI name comprising polyglycerin-6, and a third cosmetic ingredient with the INCI name comprising polyglycerin-10.

19. The cosmetic process according to claim 6, wherein the pro penetrating agent is chosen from the group consisting of:
hydrophilic molecules chosen from urea and its derivatives, glycine, trimethylglycine (betaine),
fat solubilizing agents chosen from dimethyl isosorbide and ethanol,
glycerol-based surfactants,
and mixtures thereof.

20. The cosmetic composition according to claim 12, wherein it also comprises a third cosmetic ingredient which is different from the first and from the second and comprises one or more polyglycerols which have from 3 to 10 glycerol units and have an average molecular weight ranging from 200 to 1,000 g/mol.
